(19) United States
(12) Patent Application Publication
(10) Pub. No.: US 2026/0224651 A1
(43) Pub. Date: Aug. 6, 2026

(54) PHYTO-COMPOSITIONS

(71) Applicants: Tiziana Rosa Natalina POLIMENO, Peschiera Borromeo (IT); PHYTOHEAL MEDTECH INTERNATIONAL, Dubai (AE)

(72) Inventors: Tiziana Rosa Natalina POLIMENO, Peschiera Borromeo (IT); Sebastiano Stefano Giuseppe BOGONI, Wien (AT); Daniele ANGILELLA, AREZZO (IT)

(73) Assignee: PHYTOHEAL MEDTECH INTERNATIONAL, Dubai (AE)

(21) Appl. No.: 19/155,167

(22) PCT Filed: Feb. 9, 2024
(86) PCT No.: PCT/IB2024/051229

§ 371 Date: Aug. 8, 2025

(30) Foreign Application Priority Data
Feb. 10, 2023 (IT) 102023000002271

(51) Int. Cl. A61K 36/07; A61K 8/73; A61K 8/9711; A61K 8/9728; A61K 8/9789; A61K 8/99; A61K 35/748; A61K 36/03; A61K 36/21; A61K 36/481; A61K 36/704; A61K 36/87; A61P 17/02; A61P 17/06; A61P 27/02; A61P 29/00; A61Q 17/00; A61Q 19/00

(52) U.S. Cl. CPC A61K 36/07; A61K 8/73; A61K 8/9711; A61K 8/9728; A61K 8/9789; A61K 8/99; A61K 35/748; A61K 36/03; A61K 36/21; A61K 36/481; A61K 36/704; A61K 36/87; A61P 17/02; A61P 17/06; A61P 27/02; A61P 29/00; A61Q 17/00; A61Q 19/00; A61K 2800/522; A61K 2800/591

(57) ABSTRACT

The present invention relates to a phyto-composition, having antioxidant, anti-proliferative, anti-inflammatory, regenerative and cicatrizing activity, in skin and derma. The present invention also relates to dermatological formulations comprising the phyto-composition and suitable pharmaceutically acceptable excipients. Furthermore, the present invention relates to the phyto-composition and dermatological formulations for the medical use for the treatment of human or animal dermatological disorders and/or skin lesions. Also, the present invention relates to the cosmetic use of the invention phyto-composition and dermatological formulations. Additionally, the present invention concerns an ophthalmic product comprising the phyto-composition.

### FIELD OF THE INVENTION

The present invention relates to a phyto-composition, having antioxidant, anti-proliferative, anti-inflammatory, regenerative and cicatrizing activity, in skin and derma. The present invention also relates to dermatological formulations comprising the phyto-composition and suitable pharmaceutically acceptable excipients. Furthermore, the present invention relates to the phyto-composition and dermatological formulations for the treatment of dermatological disorders and/or skin lesions. Also, the present invention relates to the use of phyto-composition and dermatological formulations as cosmetics.

### BACKGROUND ART

Skin disorders are mainly characterized by an irregular cell growth and regeneration, and/or by inflammation.

Numerous skin disorders cause hypertrophy of the horny layer of the epidermis, an event also known as hyper-keratinization. These diseases are also called ichthyosis. Typical examples of ichthyosis include Pityriasis rosea of Gibert, rosacea, seborrheic dermatitis and psoriasis.

Psoriasis is a common, long-term (chronic) disease with no cure. Although there is no wound at a psoriatic lesion, keratinocytes behave as if there is, switching from the normal growth program to regenerative maturation. When skin is wounded, a wound healing program, also known as regenerative maturation, is triggered; normal wound healing process includes a very well-orchestrated and regulated process consisting of series of events such as haemostasis, inflammation, proliferation and extracellular matrix (ECM) remodelling. Cells are produced at a much faster rate, theoretically to replace and repair the wound. There is also an increased blood supply and localized inflammation. However, in pathological conditions, including trauma wounds due to burns or accidents, this normal healing process gets severely dysregulated.

Also, at a psoriatic lesion, cells are created and pushed to the surface but the skin cannot shed the cells fast enough. The excessive skin cells build up and form elevated, scaly lesions. The white scale (called “plaque”) that usually covers the lesion is composed of dead skin cells, and the redness of the lesion is caused by increased blood supply to the area of rapidly dividing skin cells, which can be accompanied by inflammation.

These skin disorders are typically treated symptomatically with keratolysis agents to remove plaques and with glucocorticoids to relieve inflammation.

The hypertrophic skin layer may also contain infections therewithin. Therefore, antibacterial and antiseptic agents are typically used in the treatment of said disorders. Antibacterial, antiseptic and keratolytic agents, such as salicylic acid or retinoic acid, are also used to treat skin disorders caused by hormonal imbalance, such as acne, or to treat eczema and dermatitis, characterized by severe inflammation of the skin, typically accompanied by redness, swelling, oozing, scaling and itching.

Eczema can take the form of contact dermatitis or atopic dermatitis in people “atopic” or allergic by nature. If the scalp is involved the disorder is known as seborrheic dermatitis. Dermatitis can be caused by chemicals, plants, clothing fabrics, metallic materials and even medicines. Atopic dermatitis is a chronic or chronic-relapsing eczema caused by immunological and non-immunological factors. The formers consist of food allergens, inhalants or contact, the latter of external irritants, infections, neurovegetative disorders, disorders of lipid metabolism, sweating and stress. Atopic dermatitis is characterized by a reduction in the epidermal ceramides which leads to a weakening of the barrier function of the skin, increased irritability and an increase in transepidermal water loss which can increase if an epidermal inflammatory condition coexists. This facilitates the penetration into the skin of allergens and haptens that bind to keratinocytes and Langerhans cells, activating them. Skin affected by dermatitis is more susceptible to infections due to elevated levels of inflammatory cytokines.

Within the epidermis, keratinocytes are the major source of cytokines, along with Langerhans cells, dermal mast cells, dendritic cells and macrophages. While resting keratinocytes produce some cytokines constitutively, a variety of environmental stimuli, such as tumor promoters, ultraviolet light and chemical agents, can induce epidermal keratinocytes to release inflammatory cytokines (IL-1, TNF-α), chemotactic cytokines (like IL-8) and growth promoting cytokines (like IL-6 and IL-7).

Interleukin-1α (IL-1α) is constitutively produced in large amounts by keratinocytes: in normal conditions it remains stored inside them, while a damage or stimuli of various kinds can determine its release.

The primary role of IL-1α is to maintain skin barrier function by preventing the entry of pathogenic microorganisms into the body: a damage to keratinocytes releases IL-1α, which stimulates further release of IL-1α and the production and release of other cytokines such as IL-8 and IL-6. IL-1α also induces the production of several inflammatory mediators, including cyclooxygenase (COX)-2, type 2 phospholipase A and inducible nitric oxide synthase (iNOS). IL-1α is used as a marker to compare the mildness of topical formulations and to assess dermal irritation potential: mild cleansers do not upregulate or exacerbate the release of IL-1α from keratinocytes, thus preserving skin barrier integrity.

Interleukin-6 (IL-6) is a cytokine that contributes to innate immune defence. It is also essential for antibody production in B cells. In the skin IL-6 is produced constitutively and after injury by fibroblasts and keratinocytes, but can also be synthesized by monocytes and macrophages after antigen activation. After mechanical injury, IL-6 messenger RNA (mRNA) expression is upregulated in skin and exerts its effects on dendritic cells.

Interleukin-8 (IL-8) is produced by fibroblasts and keratinocytes after stimulation with proinflammatory cytokines, including IL-1α and TNF-α. IL-8 is a strong activator of chemotaxis and it may serve as a useful marker for assessing early inflammation or tissue damage in the skin.

Inflammation is then a manifestation of an abnormal skin condition but, low levels of inflammation can occur in the absence of clinical manifestations such as erythema (skin redness).

Several drugs are currently employed for treating dermatological disorders and skin lesions; however, their efficacy is incomplete and the drugs themselves can be the cause of skin reactions, especially when used for long periods. Therefore, currently available drugs not only have incomplete effectiveness, but also determine cosmetic defects.

The object of the present invention is therefore to provide a new remedy for the treatment of dermatological disorders and/or skin lesions, which is effective and well tolerated by the body and that also has positive cosmetic effects.

### SUMMARY OF THE INVENTION

Said object has been achieved by the a phyto-composition according to the invention, as set forth in the claims herein enclosed.

In another aspect, the present invention relates to dermatological formulations comprising said phyto-composition and suitable pharmaceutically acceptable excipients.

The phyto-composition and the dermatological formulations can be advantageously used as a medicament.

In particular, the phyto-composition and the dermatological formulations can be advantageously used as a medicament for the treatment of pathologies of the skin.

The phyto-composition and dermatological formulations can be used both in human and veterinary medicine.

The phyto-composition and the dermatological formulations of the invention can also have a cosmetic use.

For the purposes of the present invention, said dermatological diseases are ichthyosis, excessive secretion of sebum, microbial infection of skin and/or derma, dermatophytosis, acne, psoriasis, seborrheic dermatitis, rosacea, dandruff, alopecia, allergic dermatosis, urticaria, scleroderma, contact dermatitis, atopic dermatitis, chronic actinic dermatitis, photodermatosis; for the purposes of the present invention, said tissue lesions are maculae, papulae, vesicles, bullae, pustulae, cysts, erosions, abrasions, rashes, ulcers, chapping, sores, decubitus ulcers, telangiectasias, scales, erythema, crusts, lichenifications, excoriations, indurations, cuts, lacerations, diabetic lesions and ulcers, or burns, said lesions potentially involving also derma, further to skin. Optionally, the dermatological diseases, and/or skin lesions include the skin and/or derma defects and/or lesions due to skin oxidation.

In a further aspect, the present invention concerns an ophthalmic product comprising the phyto-composition and ophthalmologically acceptable excipients.

### BRIEF DESCRIPTION OF THE FIGURES

The characteristics and the advantages of the present invention will become clear from the following detailed description, the working examples provided for illustrative purposes and the accompanying figures, wherein:

FIG. 1 shows the dosage of IL-6 (FIG. 1A) and IL-8 (FIG. 1B) of Example 12. The absorbance measured at 450 nm is directly proportional to the quantity of interleukin produced by the tissues. The values are expressed as means±standard deviation. Statistical data processing was performed by Student's t-test. We considered significative values of p<0.05. NC=untreated and unstimulated control tissues; SDS=tissues treated with sodium dodecyl sulphate in order to stimulate cytokines production to simulate an irritation condition; TS=tissues treated with tested sample. * p<0.05 vs SDS; ** p<0.01 vs SDS.

FIG. 2 shows the results of the protein assay performed in Example 13. The absorbance values measured at 650 nm are directly proportional to the quantity of proteins produced by the cells. The values are expressed as means±standard deviation. Statistical data processing was performed by Student's t-test. p<0.05. * p<0.05 vs NC; ** p<0.01 vs NC.

FIG. 3 shows the results of the wound healing test performed in Example 14. FIGS. 3A and B respectively show the dosage of IL-6 and IL-8: the absorbance measured at 450 nm is directly proportional to the quantity of interleukins produced by the cells. The values are expressed as means±standard deviation. Statistical data processing was performed by Student's t-test. p<0.05. NC=untreated control cells. * p<0.05 vs NC; ** p<0.01 vs NC. FIG. 3C-G shows the morphological evaluation of the cellular monostrates: FIG. 3C=cut monostrate; FIG. 3D=Negative control (not treated cells); FIG. 3E-G=Sample teste at the concentration of 0.2 mg/ml, 0.1 mg/ml, 0.05 mg/ml, respectively. Magnification 10×; the dotted lines indicate the original margins of the wound.

FIG. 4 shows the quantification of PCNA immunofluorescence measure in Example 15. The values are expressed as means±standard deviation. Statistical data processing was performed by Student's t-test. p<0.05. NC=untreated control tissues; TS=tested sample; * p<0.05 vs NC; ** p<0.01 vs NC.

FIG. 5 shows the result of the cell viability tests of Example 16 on human fibroblasts and keratinocytes. The testes samples were the phyto-composition of the invention (FIG. 5A), a serum formulation comprising the same (FIG. 5B) and commercial serums (FIG. 5C).

FIG. 6 shows the results on severe psoriasis after 2 months of application.

FIG. 7 shows the results on a superficial necrosis after 15 days of application.

FIG. 8 shows the results on hematoma caused by a dog bite after 3 days of application.

FIG. 9 shows the results on severe hematoma caused by a motorcycle accident after 3 days of application.

FIG. 10 shows the results on an ocular trauma after 10 days with known treatments (A), after 3 days with the phyto-composition of the invention (B) and after 1 week with the phyto-composition of the invention (C).

FIG. 11 shows the results of the morphological observation of HaCaT cells treated 24 h with the invention phyto-compositions (A) and in fibroblasts (B) as reported in Example 18.

### DETAILED DESCRIPTION OF THE INVENTION

The phyto-composition of the invention comprises natural ingredients, that can comprise in turn several phyto-complexes.

In particular, the phyto-composition of the invention consists of the following ingredients, each in the concentration ranges by weight indicated in parenthesis (w/w, based on the total weight of the phyto-composition):
Tremella fuciformis Polysaccharide (10-20% w/w)
Vitis vinifer Leaf Extract (5-15%)
Astragalus membranaceus Root Extract (10-30%)
Medicago sativa Extract (8-15%)
Fucus vesiculosus Extract (4-15%)
Spirulia platensis Powder (4-8%)
Beta vulgaris Root Extract (8-15%), and
Polygonum cuspidatum Extract (5-30%)

Preferably, the ingredients of the phyto-composition of the invention are in the following concentration ranges by weight (w/w):
Tremella fuciformis Polysaccharide (12-16%)
Vitis vinifer Leaf Extract (8-13%)
Astragalus membranaceus Root Extract (15-25%)
Medicago sativa Extract (8-13%)
Fucus vesiculosus Extract (8-15%)
Spirulia platensis Powder (4-6%)
Beta vulgaris Root extract (8-13%), and
Polygonum cuspidatum Extract (15-25%)

For the preparation of the extracts, where not indicated, the aerial parts of the plant are used, i.e., stem, leaves, flowers or mixtures thereof. These parts can be used fresh or after drying under controlled conditions. The parts of the plant used for extracting the ingredient are contacted with a suitable extraction solvent, by using conventional extraction methods, such as maceration or percolation, or more complex techniques, such as for example extraction with ultrasound, microwaves, pressure or supercritical fluids. Examples of usable extraction solvents are alcohols having up to 4 carbon atoms, including diols and triols, aldehydes, ketones, organic esters, chlorinated compounds, and mixtures thereof. When miscible, such solvents can also be used in mixture with water. Preferred solvents include methanol, ethanol, isopropanol, butanol, ethylene glycol, propylene glycol, glycerol, acetone, ethyl acetate and mixtures thereof, as such or mixed with water.

After separation from the exhausted plant, the extract can be used as such, or after substitution of the extractive solvent with one more suitable for human use (such as glycerine or glycol, if not used in the extraction phase). Preferably, the extracting solvent is removed to give a dry extract, preferably by evaporation at reduced pressure and low temperature, and atomization.

The extract can also be subjected to subsequent purification steps, to remove potential contaminants (such as lipophilic pesticides), impurities (such as chlorophyll) or to increase the concentration of secondary metabolites.
Tremella fuciformis Polysaccharide (CAS No. 778577-37-0) is extracted from the edible fruit bodies of the Silver Ear mushroom in China. The polysaccharide comprises glucuronic acid and N-acetylglucosamine. Extracts of Tremella fuciformis can be obtained via solvent extraction from the Tremella carpophore.
Vitis vinifera Leaf Extract (CAS No. 84929-27-1) is an extract of the leaves of the Red Grape (Vitis).
Astragalus membranaceus Root Extract (CAS No. 94166-93-5) is extracted from the dried roots of the medicinal herb Astragalus membranaceus. Astragalus extracts can be obtained through an extraction process in the following order: Astragalus drying, grinding, screening and titration of individual phytocomplexes.
Medicago sativa Extract (CAS No: 84082-36-0) is an extract of the whole plant Medicago sativa (alfalfa). The extract comprises folic acid and optionally one or more of oligosaccharide, vitamin B5 e Q10 co-enzyme.
Fucus vesiculosus Extract (CAS No: 84696-13-9) is an extract of the dried thallus of the algae Bladderwrack, Fucus.
Spirulia platensis Powder (CAS No: 223751-80-2) is the powder obtained from the dried ground preferably of the whole thallus of the Arthrospira platensis, also known as Spirulina platensis (spirulina).
Beta vulgaris Root extract (CAS No: 89957-89-1/89957-90-4) is an extract of the roots of the Sugar Beet, Beta vulgaris L., obtainable by drying, extraction and titration of Beta vulgaris powders or with solvent extraction.
Polygonum cuspidatum extracts (CAS No. 501-36-0) are obtainable through a drying and titration of powders or with solvent extraction.
The Vitis vinifera Leaf Extracts and Polygonum cuspidatum extracts preferably comprise resveratrol.

Preferably, the phyto-composition according to the present invention is formulated in a dermatological formulation (also indicated herein as “dermatological product”), more preferably for topical use. Said phyto-composition is preferably present in the pharmaceutical formulation at a concentration by weight of 1-5% on the weight of the dermatological formulation; more preferably, the phyto-composition is present at a concentration of 2-4%, most preferably of about 3%, on the weight of the dermatological formulation.

The present invention is then directed also to a dermatological formulation comprising the phyto-composition above described, preferably in a concentration of 1-5%, more preferably 2-4%, most preferably of about 3%, by weight of the dermatological formulation, together with suitable pharmaceutically acceptable excipients.

Suitable pharmaceutically acceptable excipients comprise rheological additives, buffering agents, antimicrobial agents, antioxidant agents, anti-isothermal agents, antistatic agents, absorbent agents, UV absorbing agents, astringent agents, chelating agents, skin conditioning agents, preservative agents, covering agents, denaturing agents, depigmenting agents, emulsifying agents, film-forming agents, gelling agents, colloidal agents, moisturizing agents, hydrotropic agents, binders, soothing agents, smoothing agents, opacifying agents, plasticizing agents, propelling agents, skin protecting agents, reducing agents, cooling agents, sebum-restoring agents, solvents, stabilizing agents, emulsifying stabilizing agents, toning agents, wetting agents, volumizing agents or combinations thereof.

Preferably said pharmaceutically acceptable excipients comprise or consist of one or more of rheological additives, buffering agents, antimicrobial agents, antioxidant agents, emulsifying agents, moisturizing agents, hydrotropic agents, smoothing agents, solvents.

The dermatological formulations according to the invention are preferably aqueous formulations, more preferably comprising at least one alcohol-based solvent, at least one antimicrobial agent, at least one smoothing agent, at least one rheological additive, at least one antioxidant agent and/or at least one moisturizing agents.

Particularly preferred pharmaceutically acceptable excipients that are present in the dermatological formulations of the invention are: methoxymethyl butanol as alcohol-based solvent, carbomer as thickening agent, propylene glycol, polyethylene glycol (PEG) compounds, such as PEG-6, PEG-32, PEG-4, including PEG-4 Proline Linolenate and PEG-4 Proline Linoleate. Preferably, the dermatological formulation of the invention further comprises oner or more of: vitamins and/or provitamins, such as vitamin A, vitamin D, vitamin E and panthenol, preferably colloidal vitamins, such as colloidal Vitamin D, colloidal vitamin A, inorganic elements with antimicrobial activity, such as silver, copper, zinc and other antimicrobial compounds such as phenoxyethanol, 2-Bromo-2-Nitropropane-1,3-Diol.

Optionally, the dermatological formulation can further comprise glycosaminoglycans, such as chondroitin, chondroitin sulfate, dermatan sulfate, keratan sulfate, heparin, heparan sulfate, hyaluronic acid, and mixtures thereof.

The dermatological formulation may also comprise antioxidant and/or antimicrobial agents as active agents.

Therefore, the phyto-composition of the invention has shown an unexpected and surprising regenerating, reparative, anti-inflammatory and cicatrizing effect on tissue lesions of various kinds.

Accordingly, the phyto-compositions and the dermatological formulations comprising them and disclosed in the present invention are suitable for the medical use.

In particular, the phyto-compositions of the invention and the dermatological formulations comprising them are disclosed for the medical use in the treatment of dermatological diseases and skin lesions.

In a more particular aspect, the phyto-compositions of the invention and the dermatological formulations comprising them are disclosed for the medical use in the treatment of psoriasis.

In addition, the invention also relates to the cosmetic use of the phyto-compositions and of the dermatological formulations comprising them.

In particular, the invention discloses the cosmetic use of the phyto-compositions and of the dermatological formulations comprising them for the treatment of dermatological diseases and/or skin lesions.

For the purposes of the present invention, said dermatological diseases are ichthyosis, excessive secretion of sebum, microbial infection, dermatophytosis, acne, psoriasis, seborrheic dermatitis, rosacea, dandruff, alopecia, allergic dermatosis, urticaria, scleroderma, contact dermatitis, atopic dermatitis, chronic actinic dermatitis, photodermatosis. Preferably, said dermatological disease is ichthyosis, more preferably psoriasis.

For the purposes of the present invention, said tissue lesions are maculae, papulae, vesicles, bullae, pustulae, cysts, erosions, abrasions, rashes, ulcers, chapping, sores, decubitus ulcers, telangiectasias, scales, erythema, crusts, lichenifications, excoriations, indurations, cuts, lacerations, diabetic lesions and ulcers, or burns.

Formulation of said phyto-composition with specific excipients further improves the one or more of the above-mentioned dermatological advantages of the phyto-composition, so that formulation that are particularly suitable for treating specific dermatological disturbances or tissue lesions can be provided.

In particularly preferred embodiments, the dermatological formulations of the present invention comprise the phyto-composition and a mixture of methoxymethyl butanol, carbomer, propylene glycol, PEG-4 proline linolenate, PEG-4 proline linoleate, Colloidal silver, phenoxyethanol and 2-bromo-2-nitropropane-1,3-diol.

Preferred formulations having further improved anti-inflammatory activity comprise the phyto-composition together with Vitamin D and at one or more of Silver, Iron, Copper, Phenoxyethanol and 2-Bromo-2-Nitropropane-1,3-Diol; preferably vitamins, Silver and/or Iron are present as colloidal compounds.

Preferred formulations having further improved anti-oxidant, anti-bacterial, anti-inflammatory and soothing activity comprise the phyto-composition together with Vitamin A and/or panthenol, and at one or more of Silver, Zinc, Phenoxyethanol and 2-Bromo-2-Nitropropane-1,3-Diol; preferably vitamin A and/or Silver are present as colloidal compounds.

Preferred formulations having further improved anti-oxidant, anti-bacterial, anti-inflammatory and anti-acne activity comprise the phyto-composition together with sulfur, hyaluronic acid and at one or more of Silver (preferably colloidal silver), phenoxyethanol and 2-Bromo-2-Nitropropane-1,3-Diol.

Preferred formulations having further improved anti-oxidant, anti-bacterial, cicatrizing, regenerating, revitalizing and anti-psoriasis activity comprise the phyto-composition together with Vitamin E and/or panthenol, and at one or more of Silver (preferably colloidal silver), copper, phenoxyethanol and 2-Bromo-2-Nitropropane-1,3-Diol. Particularly preferred are the following dermatological Formulations 1-4.
Formulation 1 is particularly suitable for use as anti-oxidant, anti-bacterial, anti-inflammatory and soothing formulation, preferably in the form of serum:

| Formulation 1 | % (w/w) | |
|---|---|---|
| Water | >40 | |
| Methoxymethyl butanol | 5-7 | |
| Carbomer | 2-3 | |
| Propylene Glycol | 1-2 | |
| PEG-4 Proline Linolenate, | 1-2 | |
| PEG-4 Proline Linoleate | | |
| Phyto-composition | 3% | |
| Colloidal Silver (110 ppm) | 0.002 | 18.18 ML IN |
| | | AQUEOUS |
| | | SOLUTION |
| Colloidal D Vitamin | 0.003 | 6 ML IN |
| (500 ppm) | | AQUEOUS |
| | | SOLUTION |
| Colloidal Iron (20 ppm) | 0.003 | 50 ML IN |
| | | AQUEOUS |
| | | SOLUTION |
| Colloidal Copper (20 ppm) | 0.003 | 50 ML IN |
| | | AQUEOUS |
| | | SOLUTION |
| Phenoxyethanol, 2-Bromo- | 0.5-1.0 | |
| 2-Nitropropane-1,3-Diol | | |

Formulation 2 is particularly suitable for use as antioxidant, antiaging, and anti-bacterial formulation, preferably in the form of serum:

| Formulation 2 | % (w/w) | |
|---|---|---|
| Water | >50 | |
| Methoxymethyl butanol | 5-7 | |
| PEG-6, PEG-32 | 5-7 | |
| Propylene Glycol | 1-2 | |
| PEG-4 Proline Linolenate, | 2-3 | |
| PEG-4 Proline Linoleate | | |
| Carbomer | 1-2 | |
| Panthenol | 2-3 | |
| Phyto-composition | 3% | |
| Colloidal Silver (110 ppm) | 0.002 | 18.18 ML IN |
| | | AQUEOUS |
| | | SOLUTION |
| Colloidal Zinc (20 ppm) | 0.003 | 50 ML IN |
| | | AQUEOUS |
| | | SOLUTION |
| Colloidal A Vitamin | 0.003 | 6.66 ML IN |
| (450 ppm) | | AQUEOUS |
| | | SOLUTION |
| Phenoxyethanol, 2-Bromo- | 0.5-1.0 | |
| 2-Nitropropane-1,3-Diol | | |

Formulation 3 is particularly suitable for use as anti-oxidant, anti-bacterial, anti-inflammatory and anti-acne formulation, preferably in the form of serum:

| Formulation 3 | % (w/w) | |
|---|---|---|
| Water | >60 | |
| Methoxymethyl butanol | 5-7 | |
| PEG-6, PEG-32 | 10-12 | |
| Carbomer | 2-3 | |
| PEG-4 Proline Linolenate, | 2-3 | |
| PEG-4 Proline Linoleate | | |
| Propylene Glycol | 1-2 | |
| Sulfur | 0.025 | |
| Phyto-composition | 3% | |
| Colloidal Silver (110 ppm) | 0.002 | 18.18 ML IN |
| | | AQUEOUS |
| | | SOLUTION |
| Hyaluronic acid | 0.03 | 24 ML IN |
| (1'200 ppm) | | AQUEOUS |
| | | SOLUTION |
| Phenoxyethanol, 2-Bromo- | 0.5-1.0 | |
| 2-Nitropropane-1,3-Diol | | |

Formulation 4 is particularly suitable for use as anti-oxidant, anti-bacterial, cicatrizing, regenerating, revitalizing and anti-psoriasis formulation, preferably in the form of serum:

| Formulation 4 | % (w/w) | |
|---|---|---|
| Water | >60 | |
| Methoxymethyl butanol | 5-7 | |
| Carbomer | 2-3 | |
| Propylene Glycol | 2-3 | |
| Panthenol | 1-2 | |
| PEG-4 Proline Linolenate, | 1-2 | |
| PEG-4 Proline Linoleate | | |
| Phyto-composition | 3% | |
| Tocopherol | 0.5-1.0 | |
| Colloidal Silver | 0.002 | 18.18 ML IN |
| (110 ppm) | | AQUEOUS |
| | | SOLUTION |
| Colloidal Copper | 0.003 | 50 ML IN |
| (20 ppm) | | AQUEOUS |
| | | SOLUTION |
| Phenoxyethanol, 2-Bromo- | 0.5-1.0 | |
| 2-Nitropropane-1,3-Diol | | |

The dermatological formulations of the invention can be administered via topical route. Said dermatological formulations comprising the phyto-composition may be in the form of ointment, lotion, cream, emulsion, paste, gel, aqueous solution, colloid, spray, patch, serum, soaked gauze, dressing, mouthwash, or a combination thereof. Particularly preferred are formulations in the form of serum.

Preferably, said dermatological formulations are to be administered topically, on the skin, once to four-times per day, in a suitable amount. A suitable amount is e.g., an amount that can be adsorbed by massaging the formulation on the skin.

All the dermatological formulation can be prepared by methods known in the pharmaceutical technique.

In a further aspect, the present invention concerns an ophthalmic product comprising the phyto-composition and ophthalmologically acceptable excipients.

Said ophthalmic product can be a tear substitute, a collyrium, a suspension, an eye spray, a foam, an impregnated wipe, a spray patch, or a combination thereof.

The term “ophthalmologically acceptable excipient” means a compound or a mixture suitable for use in a composition for the administration to the external surface of the eye. For example, an excipient of this kind generally shall not cause an adverse reaction in the user, nor significantly inhibit, the action of the actives on the eye's surface.

Suitable excipients are antioxidants, gelling agents, sequestrants, binders, lubricants, thickening agent, tonicity regulators, filmogenic substances, and mixtures thereof.

It should be understood that all the aspects identified as preferred and advantageous for the phyto-composition are to be deemed as similarly preferred and advantageous also for the dermatological formulations, uses thereof and vice-versa.

It should be also understood that all the combinations of preferred aspects of the phyto-composition of the invention, as well as of the dermatological formulation and uses of the same, as above reported, are to be deemed as hereby disclosed.

Below are working examples of the present invention provided for illustrative purposes.

### EXAMPLES

### Example 1

A phyto-composition is prepared by mixing the following ingredients:

| Ingredient | g/100 g |
|---|---|
| Tremella Fuciformis Polysaccharide | 14 |
| Vitis Vinifer Leaf Extract | 10 |
| Astragalus Membranaceus Root Extract | 20 |
| Medicago Sativa Extract | 10 |
| Fucus Vesiculosus Extract | 11 |
| Spirulia Platensis Powder | 5 |
| Beta Vulgaris Root Extract | 10 |
| Polygonum Cuspidatum Extract | 20 |

### Example 2

A phyto-composition is prepared by mixing the following ingredients:

| Ingredient | g/100 g |
|---|---|
| Tremella Fuciformis Polysaccharide | 16 |
| Vitis Vinifer Leaf Extract | 11 |
| Astragalus Membranaceus Root Extract | 15 |
| Medicago Sativa Extract | 12 |
| Fucus Vesiculosus Extract | 10 |
| Spirulia Platensis Powder | 6 |
| Beta Vulgaris Root Extract | 8 |
| Polygonum Cuspidatum Extract | 22 |

### Example 3

A phyto-composition is prepared by mixing the following ingredients:

| Ingredient | g/100 g |
|---|---|
| Tremella Fuciformis Polysaccharide | 20 |
| Vitis Vinifer Leaf Extract | 8 |
| Astragalus Membranaceus Root Extract | 12 |
| Medicago Sativa Extract | 8 |
| Fucus Vesiculosus Extract | 11 |
| Spirulia Platensis Powder | 6 |
| Beta Vulgaris Root Extract | 10 |
| Polygonum Cuspidatum Extract | 25 |

### Example 4

A dermatological formulation has been prepared by mixing the phyto-composition of Example 2, at a final concentration of 2% by weight on the weight of the formulation (w/w), with the following excipients, in water:
an alcohol-based solvent, carbomer, PEG compounds, propylene Glycol, vitamin D and -2-Bromo-2-Nitropropane-1,3-Diol.

### Example 5

A dermatological formulation has been prepared by mixing the phyto-composition of Example 2, at a final concentration of 3% by weight on the weight of the formulation (w/w), with the following excipients, in water:
an alcohol-based solvent, carbomer, PEG compounds, propylene Glycol, panthenol, zinc and -2-Bromo-2-Nitropropane-1,3-Diol.

### Example 6

A dermatological formulation has been prepared by mixing the phyto-composition of Example 2, at a final concentration of 1.5% by weight on the weight of the formulation (w/w), with the following excipients, in water:
an alcohol-based solvent, carbomer, PEG compounds, propylene glycol, sulfur, hyaluronic acid zinc, silver, phenoxyethanol and -2-Bromo-2-Nitropropane-1,3-Diol.

### Example 7

A dermatological formulation has been prepared by mixing the phyto-composition of Example 2, at a final concentration of 2.5% by weight on the weight of the formulation (w/w), with the following excipients, in water:
an alcohol-based solvent, carbomer, PEG compounds, propylene glycol, hyaluronic acid and -2-Bromo-2-Nitropropane-1,3-Diol.

### Example 8

A dermatological formulation has been prepared by mixing the phyto-composition of Example 2, at a final concentration of 3% by weight on the weight of the formulation (w/w), with the following excipients, in water:
an alcohol-based solvent, carbomer, PEG compounds, panthenol, vitamin E, phenoxyethanol and -2-Bromo-2-Nitropropane-1,3-Diol.

### Example 9

A dermatological formulation has been prepared by mixing the phyto-composition of Example 2, at a final concentration of 3% by weight on the weight of the formulation (w/w), with the following excipients, in water:
an alcohol-based solvent, carbomer, PEG compounds, panthenol, colloidal silver, copper and -2-Bromo-2-Nitropropane-1,3-Diol.

### Example 10

The following dermatological formulations comprising the phyto-composition of Example 1 have been prepared for testing:

| Formulation 1 | % (w/w) | |
|---|---|---|
| Water | >40 | |
| Methoxymethyl butanol | 5-7 | |
| Carbomer | 2-3 | |
| Propylene Glycol | 1-2 | |
| PEG-4 Proline Linolenate, | 1-2 | |
| PEG-4 Proline Linoleate | | |
| Phyto-composition | 3% | |
| Colloidal Silver | 0.002 | 18.18 ML IN |
| (110 ppm) | | AQUEOUS |
| | | SOLUTION |
| Colloidal D Vitamin | 0.003 | 6 ML IN |
| (500 ppm) | | AQUEOUS |
| | | SOLUTION |
| Colloidal Iron (20 ppm) | 0.003 | 50 ML IN |
| | | AQUEOUS |
| | | SOLUTION |
| Colloidal Copper (20 ppm) | 0.003 | 50 ML IN |
| | | AQUEOUS |
| | | SOLUTION |
| Phenoxyethanol, 2-Bromo- | 0.5-1.0 | |
| 2-Nitropropane-1,3-Diol | | |

| Formulation 2 | % (w/w) | |
|---|---|---|
| Water | >50 | |
| Methoxymethyl butanol | 5-7 | |
| PEG-6, PEG-32 | 5-7 | |
| Propylene Glycol | 1-2 | |
| PEG-4 Proline Linolenate, | 2-3 | |
| PEG-4 Proline Linoleate | | |
| Carbomer | 1-2 | |
| Panthenol | 2-3 | |
| Phyto-composition | 3% | |
| Colloidal Silver (110 ppm) | 0.002 | 18.18 ML IN |
| | | AQUEOUS |
| | | SOLUTION |
| Colloidal Zinc (20 ppm) | 0.003 | 50 ML IN |
| | | AQUEOUS |
| | | SOLUTION |
| Colloidal A Vitamin | 0.003 | 6.66 ML IN |
| (450 ppm) | | AQUEOUS |
| | | SOLUTION |
| Phenoxyethanol, 2-Bromo- | 0.5-1.0 | |
| 2-Nitropropane-1,3-Diol | | |

| Formulation 3 | % (w/w) | |
|---|---|---|
| Water | >60 | |
| Methoxymethyl butanol | 5-7 | |
| PEG-6, PEG-32 | 10-12 | |
| Carbomer | 2-3 | |
| PEG-4 Proline Linolenate, | 2-3 | |
| PEG-4 Proline Linoleate | | |
| Propylene Glycol | 1-2 | |
| Sulfur | 0.025 | |
| Phyto-composition | 3% | |
| Colloidal Silver | 0.002 | 18.18 ML IN |
| (110 ppm) | | AQUEOUS |
| | | SOLUTION |
| Hyaluronic acid | 0.03 | 24 ML IN |
| (1'200 ppm) | | AQUEOUS |
| | | SOLUTION |
| Phenoxyethanol, 2-Bromo- | 0.5-1.0 | |
| 2-Nitropropane-1,3-Diol | | |

| Formulation 4 | % (w/w) | |
|---|---|---|
| Water | >60 | |
| Methoxymethyl butanol | 5-7 | |
| Carbomer | 2-3 | |
| Propylene Glycol | 2-3 | |
| Panthenol | 1-2 | |
| PEG-4 Proline Linolenate, | 1-2 | |
| PEG-4 Proline Linoleate | | |
| Phyto-composition | 3% | |
| Tocopherol | 0.5-1.0 | |
| Colloidal Silver (110 ppm) | 0.002 | 18.18 ML IN |
| | | AQUEOUS |
| | | SOLUTION |
| Colloidal Copper (20 ppm) | 0.003 | 50 ML IN |
| | | AQUEOUS |
| | | SOLUTION |
| Phenoxyethanol, 2-Bromo-2- | 0.5-1.0 | |
| Nitropropane-1,3-Diol | | |

### Example 11

The antioxidant activity of the tested products of Example 10 has been assessed by measuring their ability to prevent the formation of free radicals (ROS) in cell cultures of human keratinocytes.

An appropriate number of human keratinocytes (Huker) was seeded in 96-well plates and cultured in DMEM (Dulbecco's modified Eagle medium) supplemented with 10% foetal bovine serum (FBS) and 1% antibiotics (penicillin and streptomycin) and incubated at standard culture conditions (37° C., 5% CO₂). Once reached a semi-confluent monolayer, the cells were treated with scalar concentrations of tested products (dilutions 1:2 from 1.0 mg/ml) and the positive control, incubated overnight (18-22 hours) The following day the cells were washed with phosphate buffered saline (PBS) and then incubated with the DCFDA solution for 20 minutes under standard culture conditions. The DCFDA was then removed, the cells were washed twice in PBS and then irradiated with UVA for 2, 4, 6, 8 and 10 minutes in order to stimulate the production of ROS. A constant emission in the UVA range (315-400 nm) with an irradiance of 1.7 mW/cm² was assured. Untreated cells represent the negative control (NC).

At the end of each period of irradiation the fluorescence was read with a fluorimeter at a wavelength of 485 nm excitation and 530 nm emission. As positive control (PC) a substance with well-known antioxidant activity was used. An NRU assay was also performed before and after UV irradiation in order to verify that in the experimental conditions cell viability does not decrease significantly. Results of the NRU assay before UV irradiation have shown the absence of inhibition of cell viability at tested concentrations between 0.0156 and 1.0 mg/ml. So the ROS dosage was performed on these concentrations.

All tested formulations showed antioxidant activity (significative % reduction of ROS) after UV exposure.

The formulations of the invention are thus suitable for counteracting and delaying the process of skin aging due to oxidative stress.

### Example 12

The soothing activity of Formulation 1A of Example 10 has been tested using a model of Reconstructed Human Epidermis (RHE), consisting of normal human keratinocytes cultured on an inert polycarbonate filter at the air-liquid interface. It represents a highly differentiated and stratified epidermis model comprising the main basal, supra basal, spinous and granular layers and a functional stratum corneum. The RHE model presents a histological morphology, composition and aspects of biochemistry comparable to the in vivo human tissue, so it is highly representative and predictive of the effects of the product once used in vivo.

Dosage of cytokines was performed on three sets of at least 3 tissues: one set of 3 tissues didn't receive any treatment (NC, negative control), a second set of 3 tissues was treated with sodium dodecyl sulphate (SDS) to stimulate the production of interleukins, while the third set of 3 tissues was treated with SDS and with the tested sample (TS) as it is, in order to verify whether the sample is able to reduce the production of interleukins (soothing activity). After an incubation period of 60 minutes, the tissues were washed to remove all traces of treatment. After 24 hours the medium was collected for the dosage of the interleukins. The interleukin content in the culture medium was determined by ELISA (enzyme-linked immunosorbent assay), a plate-based assay technique designed for detecting and quantifying substances such as peptides, proteins, antibodies and hormones. The colorimetric reaction is proportional to the amount of cytokine present in the medium. The results were read using a spectrophotometer at 450 nm. To calculate the interleukin concentrations, the OD values were interpolated into a standard interleukin curve. The percentages were calculated with respect to unstimulated and untreated tissues (NC).

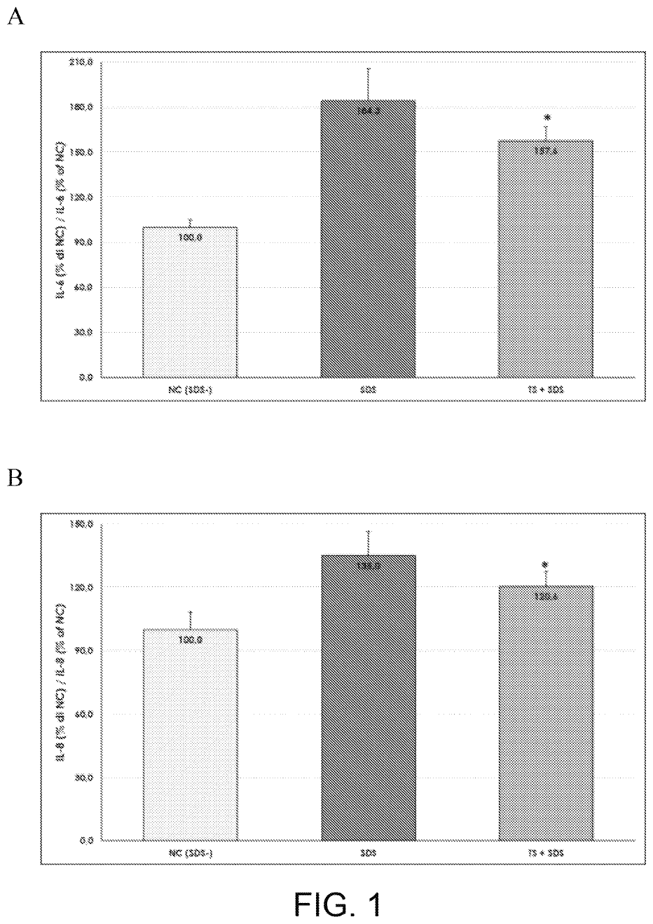

As shown in FIG. 1, the tested product has proved to significantly reduce IL-6 (protective activity of 14.5%, with respect to untreated tissues) and IL-8 (protective activity of 10.8%, with respect to untreated tissues).

These results demonstrate the soothing effect of the formulation tested, supporting its use in the treatment of dermatological disorders.

### Example 13

The regenerating activity of Formulation 4A of Example 10 has been tested by measuring its ability to induce the production of proteins in cell cultures of huma fibroblasts.

The test was performed on human fibroblasts (NHDF) cultured in DMEM (Dulbecco's modified Eagle medium) supplemented with 10% foetal bovine serum (FBS) and 1% antibiotics (penicillin and streptomycin) and incubated at standard culture conditions (37° C., 5% CO₂).

The cells were seeded and treated with concentrations of the tested product of 0.2, 0.1 and 0.05 mg/ml, chosen on the basis of a preliminary cytotoxicity test (not shown). One series of cells was not treated and maintained in medium (NC, negative control); one series was treated with a substance with a well-known regenerating activity (PC, positive control). At the end of 24, 48 and 72 hours of contact, the cells were lysed to recover the cytoplasmic content on which the measurement of the total proteins is carried out. Proteins were dosed by Lowry assay. Percentages are calculated using the absorbance values at 650 nm and considering as 100% the OD of untreated cells (NC).

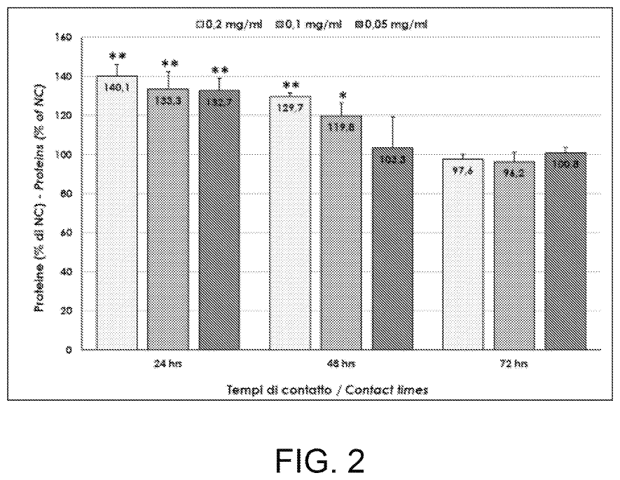

As shown in FIG. 2 the tested product has proved to significantly increase the protein content at tested concentrations of 0.2 and 0.1 mg/ml after 24 and 48 hours of contact and at tested concentration of 0.05 mg/ml after 24 hours of contact, demonstrating its regenerative effect and supporting its use for treating skin lesions.

### Example 14

The wound healing activity of Formulation 4A of Example 10 has been tested by evaluating the ability of the tested product to accelerate the rapprochement of the flaps of a wound simulated by cutting a cell monolayer of human fibroblasts.

The test was performed on human fibroblasts (NHDF) cultured in DMEM (Dulbecco's modified Eagle medium) supplemented with 10% foetal bovine serum (FBS) and 1% antibiotics (penicillin and streptomycin) and incubated at standard culture conditions (37° C., 5% CO₂).

The cells were seeded and maintained in standard culture conditions until a confluent monolayer was achieved. At this point, we performed a cut of the monolayer in order to simulate a wound and the cells were treated with the product concentrations of 0.2, 0.1 and 0.05 mg/ml chosen on the basis of the results of a preliminary cytotoxicity test not shown). Untreated cells maintained in medium represent the negative control (NC). A series of cells was treated with a positive control with a known wound repair stimulating action (PC). The cells were monitored under an optical microscope to assess any reduction or closure of the cut with the approach of the wound edges. Bright-field images are acquired under an optical microscope and the IL-1α assay is performed.

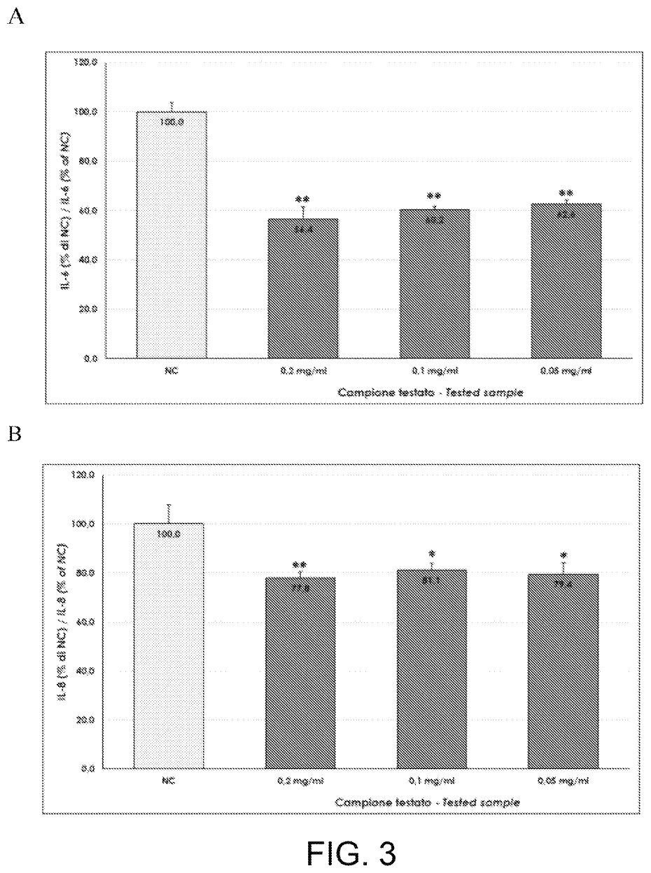

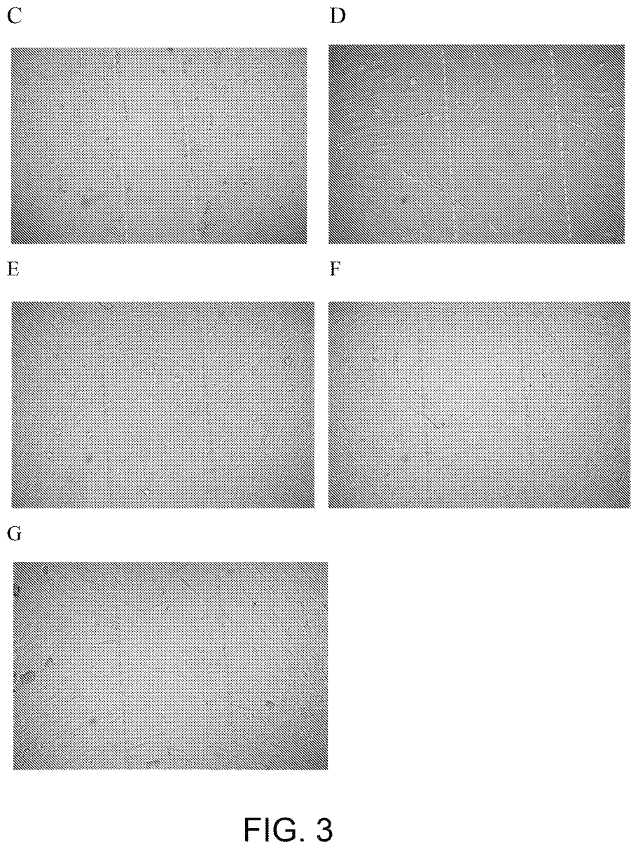

After taking the photos, the culture media in which the cells have been incubated are collected to perform the ILs assay. The content of ILs in the culture medium was determined by ELISA (enzyme-linked immunosorbent assay). To calculate the interleukin concentrations, the OD values were interpolated into a standard interleukin curve. The percentages were calculated with respect to untreated cells (NC). As shown in FIGS. 3A and B, the tested product significantly reduces IL-6 and IL-8 at all the tested concentrations. As shown in FIG. 3C-G, an approximation of the edges of the cut was observed in the cells treated with tested sample at each concentration after 24 hours of contact. The results obtained show that the formulation tested is able to stimulate the cell repairing processes in cell cultures of human fibroblasts, demonstrating a cicatrizing and regenerative activity that support its use in the treatment of skin lesions.

### Example 15

The revitalizing activity of Formulation 4A of Example 10 has been tested by measuring its ability to induce the expression of Proliferating cell nuclear antigen (PCNA) in a model of human reconstructed epidermis (RHE). PCNA plays an essential role in nucleic acid metabolism as a component of the replication and repair machinery. An increase in the expression of this marker suggests a revitalizing activity of a tested product.

The test was performed using a model of Reconstructed Human Epidermis (RHE).

The tissues were treated for 1 and 4 hours with the tested product. Untreated tissues represent the negative control (NC). At the end of the contact times, the tissues were washed in phosphate buffer (PBS) and then fixed in 4% paraformaldehyde, embedded in paraffin and cut into microtome sections. The tissue sections, after being deparaffinized with an alcohol scale, were washed in PBS and then subjected to immunolabeling for PCNA. The nuclei were stained with Hoechst 33258. The preparations were mounted in a drop of Mowiol for fluorescence microscopy.

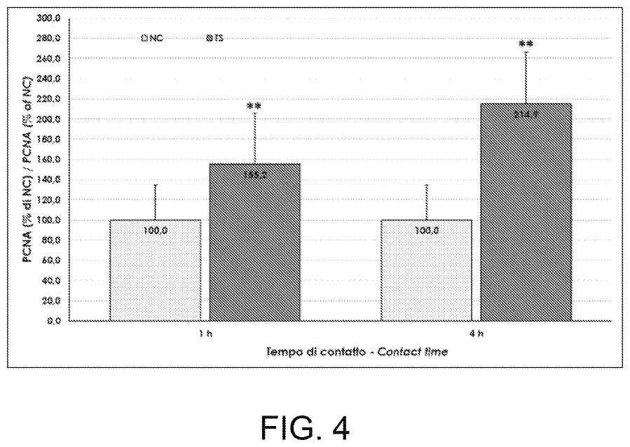

Following the immunofluorescence reaction, image acquisition was carried out using the Cell F software. The ImageJ software was used for the quantitative analysis of the images. The percentages reported were calculated based on the measured fluorescence values and the percentage of PCNA compared to the untreated tissues (NC). As shown in FIG. 4, both after 1 hour and after 4 hours of contact the immunofluorescence for PCNA increases (+55.2%, after 1 hour, which reaches +114.9% after 4 hours of contact). The results obtained demonstrate that the tested product has revitalizing activity on reconstructed human epidermis, supporting its use for treating dermatological diseases and skin lesions.

### Example 16

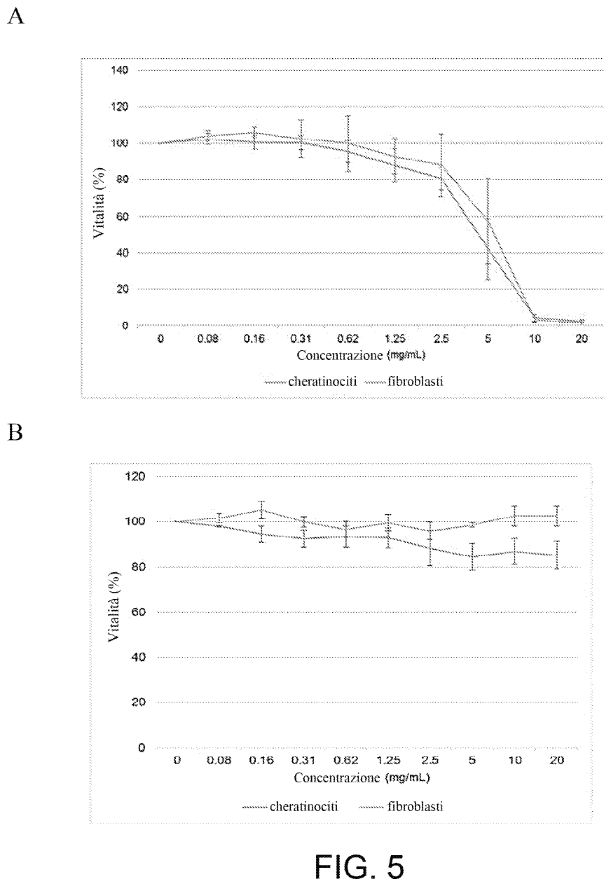

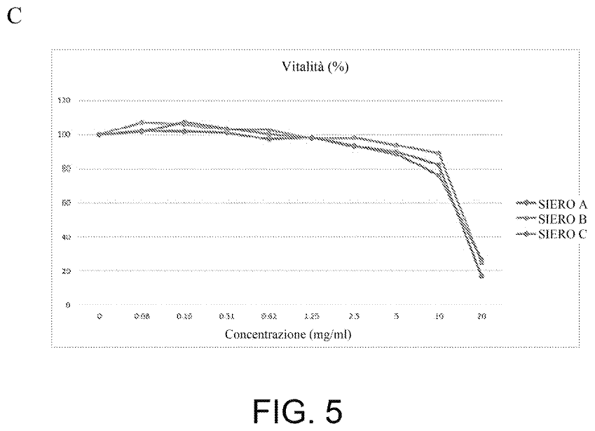

Safety of the phyto-composition of the invention as such or formulated in the form of serum for topical administration were evaluated. The compounds were tested in vitro using a cell model consisting of two human cell lines: fibroblasts of the dermis and keratinocytes of the epidermis, which together constitute the skin tissue. Either the phyto-composition (FIG. 5A) and the serum (FIG. 5B) were highly tolerated by both human skin cell lines after direct administration in the culture medium.

Even at the highest doses included in the model, no reduction in cell viability exceeding 20% is observed as a potential indicative threshold of cellular distress. Compared to other commercial topical serums (FIG. 5C), which were demonstrated to be toxic at concentrations higher than 10 mg/ml, the tested serum according to the invention could be administered to cells at the double concentration of 20 mg/ml without reducing their vitality, thus highlighting a high safety profile.

### Example 17

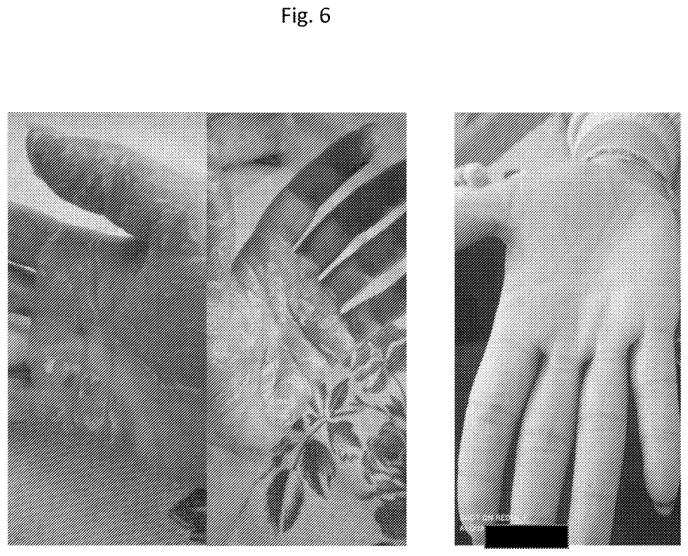

The enclosed FIGS. 6 to 10 show the surprising effects offered by the compositions of the invention after ordinary treatments failed or did not provide any satisfying effect.

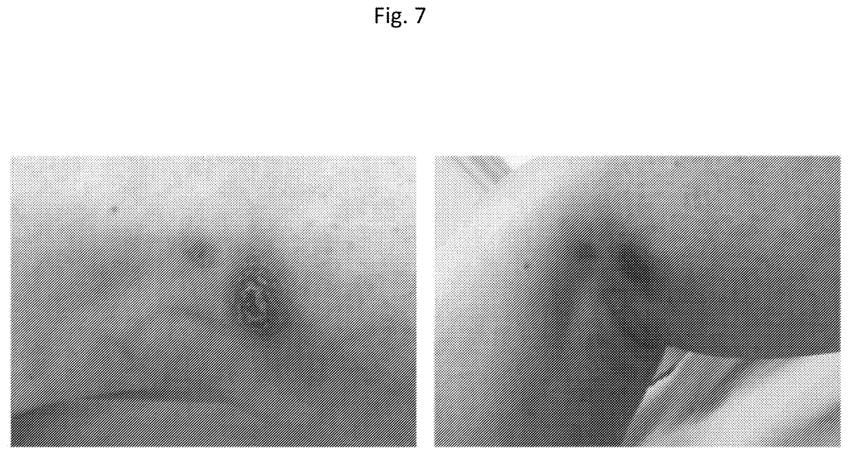

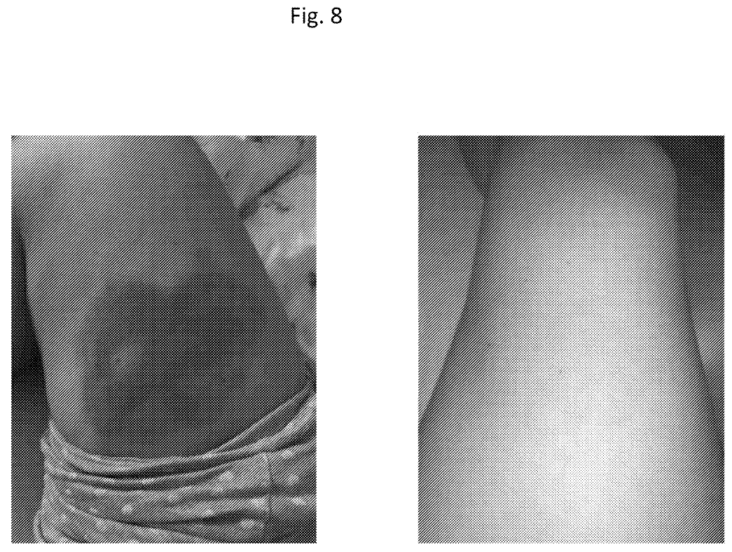

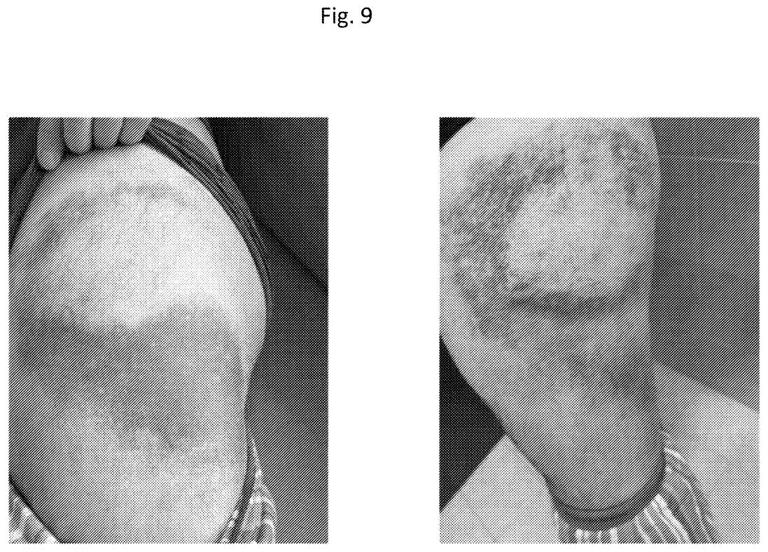

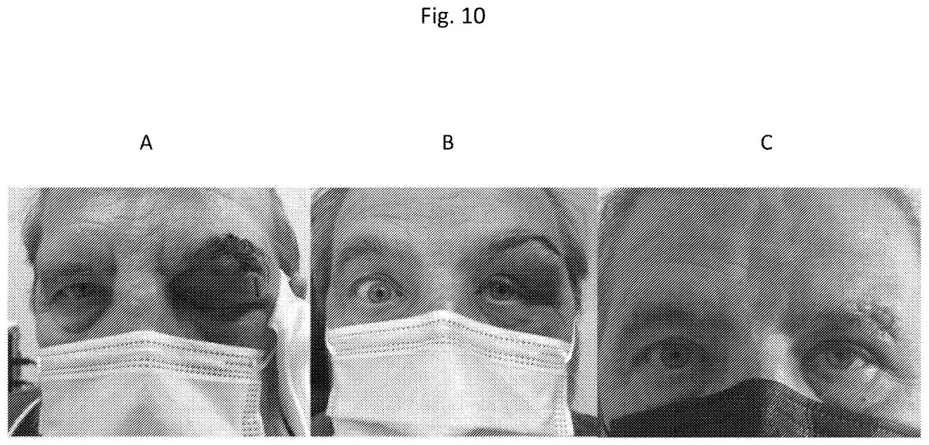

In particular:
FIG. 6 shows the results on severe psoriasis after 2 months of application.
FIG. 7 shows the results on a superficial necrosis after 15 days of application.
FIG. 8 shows the results on hematoma caused by a dog bite after 3 days of application.
FIG. 9 shows the results on severe hematoma caused by a motorcycle accident after 3 days of application.
FIG. 10 shows the results on an ocular trauma after 10 days with known treatment (A), after 3 days with the phyto-composition of the invention (B) and after 1 week with the phyto-composition of the invention.

### Example 18

The phyto-composition of the invention are suitable for the treatment of psoriasis

### Method Used and Operating Principle.

Flow cytometric analysis of bromodeoxyuridine (BrdU) as a proliferation marker, associated with propidium iodide (IP) labeling was evaluated as functional parameter to investigate the distribution of cells in the phases of the subsequent replicative cycle treatment with the mix of phytocomplexes. At the end of the treatments for 24 hours with the phytocomplexes according to the usual methods, BrdU (20 μM), a thymine analogue, was administered directly to the cells in the culture medium for 1.5 h with the aim of allowing its incorporation into the DNA of active cells proliferation. The cells were then fixed with 70% ethanol and stored at −20° C. until further processing immuno-labeling. After bringing the cells back to room temperature, acid hydrolysis was carried out of DNA and its denaturation through treatment with 2N HCl and subsequent treatment with sodium tetraborate (NA2B4O7), washing and resuspension of cells in PBS+0.2% Tween 20 and 1% albumin. After 30 minutes, the game began labeling with primary anti-BrdU antibody (DaKo) by incubation for 2 h. After appropriate washing, yes proceeded with incubation with FITC-conjugated secondary antibody for 30 min and, subsequently, with labeling with IP (solution containing Propidium Iodide, Nonidet and Ribonuclease A in PBS) for 30 min at Room temperature and then overnight at 4° C. The samples were finally read on the Attune™ NxT Acoustic flow cytometer Focusing Cytometer (Invitrogen, US). Biparametric analysis of BrdU associated with the IP marking; BrdU, in fact, is incorporated into the DNA during replication, while IP is a DNA intercalator typically used to label the nucleic acid content of cells.

### Results

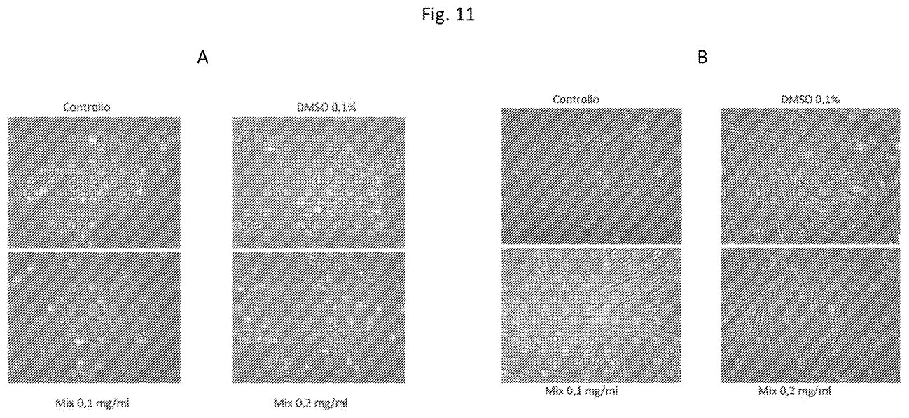

Morphological observation of HaCaT cells treated for 24 h with the two concentrations of the mix of phytocomplexes found to be non-cytotoxic, highlights a reduced cell growth underlined by the absence of mitosis (visible in the FIG. 11A obtained via phase contrast microscope as small circles) and, at the highest concentration, by a morphological modification towards a more mesenchymal phenotype.

In more detail, the proliferative capacity of these cells was investigated using flow cytometry, in association with their distribution in the cell cycle (see FIGS. 11A and 11B). In the images there are shown the Representative cytograms obtained from BrdU labeling as a function of cell distribution in the replicative cycle.

In control and solvent control cells the characteristic distribution of BrdU to iron is observed crotch of actively proliferating cells; in particular the cloud above indicates cells that are in S phase. Also the distribution of cells in the cycle replicative highlights a significant S phase.

In the presence of a mix of phytocomplexes, at concentrations of 0.1 and 0.2 mg/ml, the cloud corresponding to the S phase it flattens and the cells appear blocked in G1; similarly the DNA profile has a distribution corresponding. From the results it is not possible to establish whether the inhibitory response could be dose dependent.

Similarly to what was highlighted in HaCaT, in fibroblasts the morphological observation at 24 hours of treatment highlights once again a clear disappearance of mitosis following the treatments performed. On the contrary, these are clearly evident in control cells. Flow cytometric analysis highlights a cloud of BrdU positive cells that is more modest than to HaCaT cells, consistently with the less proliferative cell type, and again a block in G1 after treatment with a mix of phytocomplexes at respective concentrations of 0.1 and 0.2 mg/ml.

The distribution of cells in the replicative cycle also highlights the block in G1 with a marked reduction of cells in S phase.

### CONCLUSIONS

Overall, it is possible to state that the treatment with mixes at concentrations of 0.1 and 0.2 in HaCaT cells and fibroblasts it induces antiproliferative activity with a confirmation of the blockade of replicative synthesis of the hypothesis advanced in the introduction to this investigation.

1. A phyto-composition consisting of:
10-20% (w/w) of Tremella fuciformis Polysaccharide
5-15% (w/w) of Vitis vinifer Leaf Extract
10-30% (w/w) of Astragalus membranaceus Root Extract
8-15% (w/w) of Medicago sativa Extract
4-15% (w/w) of Fucus vesiculosus Extract
4-8% (w/w) of Spirulia platensis Powder
8-15% (w/w) of Beta vulgaris Root Extract, and
5-30% (w/w) of Polygonum cuspidatum Extract

2. The phyto-composition of claim 1, consisting of:
12-16% (w/w) of Tremella fuciformis Polysaccharide
8-13% (w/w) of Vitis vinifer Leaf Extract
15-25% (w/w) of Astragalus membranaceus Root Extract
8-13% (w/w) of Medicago sativa Extract
8-15% (w/w) of Fucus vesiculosus Extract
4-6% (w/w) of Spirulia platensis Powder
8-13% (w/w) of Beta vulgaris Root Extract, and
15-25% (w/w) of Polygonum cuspidatum Extract

3. A formulation comprising the phyto-composition of claim 1, and pharmaceutically acceptable excipients.

4. The formulation of claim 3, wherein the phyto-composition is in a concentration of 1-5% by weight, on the weight of the dermatological formulation, preferably 2-4% by weight.

5. The formulation of claim 3, further comprising at least one vitamin selected from Vitamin A, vitamin E, vitamin D, panthenol, and mixtures thereof, and/or at least one anti-microbic agent selected from silver, sulfur, copper, phenoxyethanol, 2-Bromo-2-Nitropropane-1,3-Diol and mixtures thereof.

6. The formulation of claim 3, further comprising at least one glycosaminoglycan, preferably hyaluronic acid.

7. The formulation of claim 3, in the form of ointment, lotion, cream, emulsion, paste, gel, aqueous solution, colloid, spray, patch, serum, soaked gauze, dressing, mouthwash, or a combination thereof, preferably in the form of serum.

8. The formulation of claim 3, which is a dermatological formulation.

9. The phyto-composition of claim 1 for use as a medicament.

10. The phyto-composition of claim 1 for use as a medicament in the human or in the veterinary field.

11. The phyto-composition of claim 1 for use as a medicament in the treatment of dermatological diseases and/or skin lesions.

12. An ophthalmic product comprising the phyto-composition of claim 1 and ophthalmologically acceptable excipients, said ophthalmic product being a tear substitute, a collyrium, a suspension, an eye spray, a foam, an impregnated wipe, a spray patch, or a combination thereof.

13. Cosmetic use of the phyto-composition of claim 1.
